(19) United States
(12) Patent Application Publication
(10) Pub. No.: US 2013/0267837 A1
(43) Pub. Date: Oct. 10, 2013

(54) ELECTRICAL LEAD POSITIONING SYSTEMS AND METHODS

(71) Applicant: NEUROACCESS TECHNOLOGIES, Boston, MA (US)

(72) Inventors: Gregory Schulte, Minneapolis, MN (US); Scott Kokones, Brookline, MA (US); Shivanand Lad, Durham, NC (US); Vincent Owens, Hingham, MA (US); Andrew Firlik, Darien, CT (US)

(73) Assignee: NeuroAccess Technologies, Boston, MA (US)

(21) Appl. No.: 13/832,063

(22) Filed: Mar. 15, 2013

Related U.S. Application Data
(60) Provisional application No. 61/622,337, filed on Apr. 10, 2012.
(60) Provisional application No. 61/712,517, filed on Oct. 11, 2012.
(60) Provisional application No. 61/729,452, filed on Nov. 23, 2012.

(51) Int. Cl. A61N 1/36; A61B 6/00; A61B 6/12

(52) U.S. Cl. CPC A61N 1/36071; A61B 6/12; A61B 6/487
USPC 600/424; 607/116; 607/117; 607/46

(57) ABSTRACT

The present invention provides systems, devices and methods for positioning a lead at a target site in a patient's body. Electrical lead positioning systems include positioning devices that aid in positioning electrodes of an electrical lead closer to the therapy site.

### CROSS-REFERENCE TO RELATED APPLICATIONS

The present application claims priority to U.S. Provisional Application No. 61/622,337 filed on Apr. 10, 2012 entitled: “Systems and Methods for Stimulating the Spinal Cord;” U.S. Provisional Application No. 61,712,517 filed on Oct. 11, 2012 entitled: “Lead Assemblies, Implantation Tools, Accessory Tools, and Systems and Methods of Using Same;” and U.S. Provisional Application No. 61/729,452 filed on Nov. 23, 2012 entitled: “Positioning Elements and Tools for Adding Positioning Elements to Leads and Catheters,” all of which are incorporated by reference herein in their entirety.

### TECHNICAL FIELD

The present invention relates to devices, systems and methods for positioning an electrical lead in a patient's body.

### BACKGROUND

Spinal cord stimulation (SCS) has been used to treat a wide variety of disorders including chronic pain, phantom limb pain, ischemic limb pain, and pain after failed spinal surgery. Physicians routinely use two different SCS systems: those involving percutaneously placed electrical leads and those requiring laminectomies to allow placement of an electrical lead. The first system involves percutaneous insertion of an electrical lead into the epidural space and either transcutaneous connection to an external generator, allowing a trial period of stimulation, or subcutaneous connection to an implanted radio frequency-controlled receiver or an implantable pulse generator. The second system requires implantation of a paddle-type electrical lead into the epidural space after a laminectomy. Similar to percutaneously placed lead, the electrical paddle-type lead may be connected to an external generator, allowing a trial period of stimulation, or may be connected subcutaneously to a radio frequency receiver or an implantable pulse generator. The radio frequency receiver is activated by an external battery-powered transmitter, which operates through an antenna placed over the receiver. The implantable pulse generator contains a battery that supplies power to the electrodes of the lead. (See Tracy Cameron, “Safety and efficacy of spinal cord stimulation for the treatment of chronic pain: a 20 year literature review,” J. Neurosurg (Spine 3), 100:254-267 (2004)).

The exact anatomical location of SCS lead placement depends on the location of the painful region. SCS leads have been placed in locations from C-1 to L-5 to treat pain of the trunk and/or limb. To achieve optimal pain relief effects, stimulation paresthesias should cover the area of pain. (See Cameron, page 254). As such, it is important that the electrodes of the electrical lead be properly positioned in the spinal canal in order to achieve optimal therapeutic benefits.

### SUMMARY OF THE INVENTION

The present invention provides systems, devices and methods for positioning a lead at a target site in a patient's body. For example, in certain embodiments, the present invention provides a positioning device that is separate or integral with an electrical lead to assist in lead positioning and to urge electrodes of the electrical lead towards the therapy site. In a preferred embodiment, the positioning device is a separate device that is added to an electrical lead by a user prior to insertion of the lead in the patient's body. In other embodiments, the device is integrated into the structure of the lead. Although the positioning device is described herein with references to a percutaneous SCS lead, the positioning device can be used with other leads such as paddle-style leads or irregularly shaped leads. Additionally, a positioning device can be used for leads and catheters that are not designed for use in SCS, such as, for example, deep brain leads, gastric leads, vagus nerve or other cranial nerve leads, intradural leads, spinal nerve leads, peripheral leads, and drug delivery catheters.

In a preferred embodiment, a positioning device comprises arms, such as axial protrusions, extending from the body of an electrical lead. The arms are flexible enough to fold against the lead body during passage through a needle or other delivery device to the target site and not cause damage to tissue, such as spinal tissue. However, the arms are stiff enough to offer resistance to tissue surrounding the target site such as surrounding tissue in the epidural space. In a preferred embodiment, the angle between each of the arms and the longitudinal axis of the lead body or a hub disposed about the lead body (as described below) is greater than 100 degrees. This assists with passage through a touhy-style needle, which is the popular insertion method for SCS percutaneous leads, as well as forward passage into the epidural space. Such an angle also adds anchoring ability and resistance to backward movement of the lead assembly. In certain embodiments, the arms provide greater resistance in one direction of movement than the other. For example, the lead can be easy to push forward to the desired target because the arms move more easily against the lead body. However, when the lead is retracted or pulled, the arms offer greater resistance. In a preferred embodiment, a positioning device offers greater resistance to pulling, but not a resistance that is so high it cannot be overcome or would cause damage, if it is desired to remove the lead.

Although a positioning device of the present invention can assist with lead anchoring, a positioning device primarily functions to position the electrodes closer to the target site. In a preferred embodiment, the arms of a positioning device are long enough such that they are at minimum the size of the passage in which a lead is inserted. It may be beneficial for the arms to be longer than that of the passage diameter, as this places more resistance against the passage walls. The arms can be manufactured of any reasonable length, and may be trimmed to an appropriate size as determined by the implanting physician. Alternatively, the arms may be manufactured in various sizes.

Preferably, the electrodes of a SCS lead make intimate contact with the dura. Often, a problem known in the art as “windshield wipering,” occurs when the SCS distal tip moves into the left or right gutter of the spinal cord space. In a preferred embodiment, a positioning device comprises a plurality of arms that protrude in a minimum of three directions. In a preferred embodiment, looking directly at a cross-section of the spinal cord with the dorsal side at zero degrees, two of the three arms oppose each other at approximately 90 and 270 degrees to resist lateral movement of the distal electrodes into the epidural canal or intradural canal. The third arm preferably is positioned at approximately zero degrees to resist dorsal movement and force the electrodes into intimate contact with the dura. The intimate contact and increased stable positioning offers the benefit of lower energy consumption due to decreased electrical resistance, and less changes to optimal stimulation settings due to decrease unintended stimulation and paraesthesia migration. The arms of a positioning device, however, are not limited to three, as there can be more, which allows the arms to provide resistance to lead movement in different directions.

Additionally, the arms of a positioning device may fold back on themselves, or take several different shapes when relaxed into their natural manufactured position. This may offer increased force against the passage walls, thereby increasing anchoring force and resistance to movement. In a preferred embodiment, the arms may still be stretched out along the lead body and keep the lead profile to a maximum outer diameter that would allow passage through an introducing needle.

The positioning device may comprise multiple axial sets of arms located on the distal end of an electrical lead. A minimum of one set may be used, up to multiple sets between axial electrodes and even extending proximally onto the mid-lead body. Again, the arms of a positioning device may be incorporated into the lead structure or added by means of a secondary device at the time of surgery or even beforehand in a secondary manufacturing phase.

In order to keep the arms from applying outward axial force before it is desired, certain embodiments provide for a thin sheath that may be place on the outside diameter of the lead and over the positioning device. Once the lead distal electrodes are in their desired location or past a point where the arms may be extended, the sheath can be retracted by the user, allowing the arms to apply outward axial force. Additionally, the sheath may be a coating or a material that is bioabsorbable such that the arms are exposed over time once implanted.

In a preferred embodiment, the positioning device (whether built into the lead or added at a later point) is radiopaque for visibility by the physician while using fluoroscopy to place the lead. If elastomeric, the positioning device may be loaded with a material such as barium to increase radiopacity. In a preferred embodiment, only part of the positioning device is radiopaque. This allows visualization of the arms under fluoroscopy and would also allow the user to determine rotational orientation if the radiopaque loading or markings are asymmetrical on the lead body. If there are three arms spanning 180° about the lead's outer diameter as described above in a preferred embodiment, there is rotational asymmetry that is desirable to visualize and could be done so by an asymmetrical radiopaque marking such as a dot or tag on only one side of the device.

In embodiments where the arms are part of an addable device that is assembled onto the lead, it is preferable to provide extra holding force to the lead outer diameter. This may be accomplished through features built into the electrical lead design, a suture, adhesive, or by using elastic material such as silicone, polyurethane, or a silicone—polyurethane blend that is undersized to the lead outer diameter so that an interference fit provide holding force between the arms and the electrical lead.

In general, an addable positioning device can have an elastic hub with an inner diameter smaller than the lead's outer diameter. The arms can be fabricated of any suitable biocompatible material, rigid or elastic. In a preferred embodiment, the hub and arms of a positioning device are one device that has been molded from an elastic silicone material. This allows the arms to collapse as the lead assembly is being placed through an introducer such as an implantation device and implanted in its final position.

Regarding particular embodiments of the present invention, in an embodiment, the present invention provides an electrical lead system for positioning an electrical lead at a target site of a patient's body. The system comprises an electrical lead, an electrical conductor and a positioning device. The electrical lead comprises an electrical lead body having a proximal portion with a proximal end, a distal portion with a distal end, and a length between the proximal and distal ends. The length has a longitudinal axis. The electrical lead further comprises an electrode array comprising a plurality of electrodes. The electrode array is located on the distal portion of the lead body. The system further comprises an electrical conductor extending between the proximal portion and the distal portion of the lead body and in electrical communication with the electrode array. The system further comprises
a positioning device on the lead body, the positioning device comprising at least three arms radiating from the lead body, wherein the angle between at least two of the arms is greater than 120 degrees.

In another embodiment, the present invention provides a method of positioning an electrical lead in a target site of the spinal canal of a patient. The method comprises providing an electrical lead system. The electrical lead system comprises an electrical lead including an electrical lead body having a proximal portion with a proximal end, a distal portion with a distal end, and a length between the proximal and distal ends. The electrical lead also includes an electrode array comprising a plurality of electrodes located on the distal portion of the lead body. The electrical lead system further comprises an electrical conductor extending between the proximal portion and the distal portion of the lead body and in electrical communication with the electrode array. The electrical lead system also includes a positioning device mountable on the lead body. The positioning device comprises at least three arms radiating from the lead body, wherein the angle between at least two of the arms is greater than 120 degrees. The method further comprises using an installation tool to mount a positioning device on the lead body. The method also includes inserting the electrical lead into the patient's body and placing the electrical lead in the patient's spinal canal. The lead can be inserted percutaneously. The plurality of arms of the positioning device extends radially outward away from the lead body and offers resistance to tissue in the spinal canal and directs at least one of the electrodes of the electrode array towards the spinal cord.

### BRIEF DESCRIPTION OF THE DRAWINGS

FIG. 1 is a perspective view of an electrical lead system according to an embodiment of the present invention.

FIG. 2 is a perspective front view of a positioning device of an electrical lead system according to an embodiment of the present invention.

FIG. 3 is a perspective back view of a positioning device of an electrical lead system according to an embodiment of the present invention.

FIG. 4A is a schematic illustration of an electrical lead system implanted in the spinal canal according to an embodiment of the present invention. FIG. 4B is a schematic transverse view of the spinal column with an electrical lead positioning system positioned against the dura of the spinal canal and the tissue surrounding the spinal canal according to an embodiment of the present invention.

FIG. 5A is a schematic transverse view of the spinal column with an electrical lead positioning system positioned against the dura of the spinal canal and the tissue surrounding the spinal canal according to an embodiment of the present invention.

FIG. 5B is a schematic transverse view of the spinal column with an anchoring device having three protrusions spaced 120 degrees apart positioned in the spinal canal.

FIG. 6 is a perspective view of an electrical lead system according to an embodiment of the present invention.

FIG. 7 is a perspective view of a positioning device of an electrical lead system according to an embodiment of the present invention.

FIG. 8 is a perspective view of a positioning device of an electrical lead system according to an embodiment of the present invention.

FIG. 9 is a perspective front view of an anchoring device of an electrical lead system according to an embodiment of the present invention.

FIG. 10 is a perspective view of an insulating device according to an embodiment of the present invention.

FIG. 11 is a perspective view of an insulating device in a rolled configuration according to another embodiment of the present invention.

FIG. 12 is the insulating device of FIG. 11 in a unrolled configuration.

FIG. 13 is a fluoroscopic marker system according to an embodiment of the present invention.

FIG. 14 is a schematic illustration of a patient's back with fluoroscopic labels of a fluoroscopic marker system adhered to the patient's back according to an embodiment of the present invention.

### DETAILED DESCRIPTION

The present invention provides systems, devices and methods for positioning a medical device at a target site in a patient's body. In preferred embodiments, the patient is a mammal, such as a human being. In certain embodiments, the medical device delivers a therapy signal to a therapy site in the patient's body. The therapy site can be the same site as the target site or can be adjacent to the target site. The medical device, for example, can be an electrical lead and/or a drug delivery catheter and the therapy signal can be an electrical signal and/or a chemical signal that provides a therapeutic effect to the patient. Although the present invention may be described with respect to SCS where the therapy site is the spinal cord and the target site is the epidural or intradural space of the spinal canal, the present invention can be used for other therapeutic purposes for other parts of a patient's body. For example, the present invention can be used for neuromodulation or other therapies of the brain, including the cortex and specifically the motor cortex; cranial nerves such as the vagus nerve; peripheral nerves such as spinal nerves including the occipital nerve and sacral nerve; and other regions of the nervous system, both the central and peripheral nervous system. The systems, devices, and methods can be used for deep brain leads, gastric leads, vagus nerve leads, peripheral nerve leads including occipital nerve and sacral nerve leads, drug delivery catheters, cardiac catheters, and cardiac stimulation leads.

The disclosure herein may refer to electrical or neural “stimulation” or “modulation.” Such terms include inhibition or activation of electrical activity in and/or around the therapy site. The terms “laterally,” “anteriorly” and “posteriorly” are used herein with respect to the anatomical directions of a human body in a standard anatomical position as is known in the art. The disclosure herein also refers to the term “substantially” with respect to certain geometric shapes and configurations. By “substantially” is meant that the shape or configuration of the described component, feature, or element need not have the mathematically exact described shape or configuration, but can have a shape that is recognizable by one skilled in the art as generally or approximately having the described shape or configuration. Also, the disclosure herein refers to an “operative configuration.” This is the configuration of the system when the medical device has been inserted into the patient and is being steered to the target site. Further, as used herein with respect to a described component, the terms “a,” “an,” and “the” include at least one or more of the described component, feature or element unless otherwise indicated. Moreover, the term “or” includes the term “and/or” unless otherwise indicated. In addition, it will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

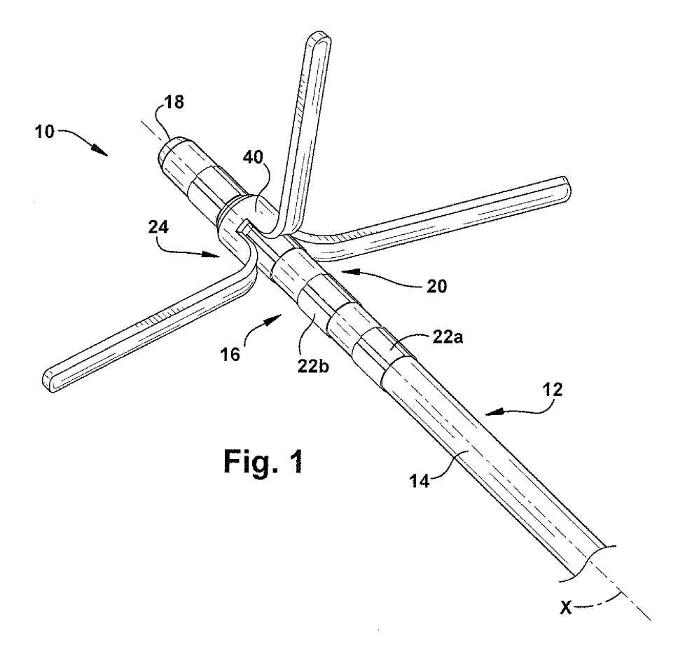

Referring to FIG. 1, the present invention provides an electrical lead system 10 comprising an electrical lead 12 comprising an electrical lead body 14 having a proximal portion (not shown) with a proximal end (not shown), a distal portion 16 with a distal end 18, and a length between the proximal and distal ends. The length has a longitudinal axis X. Electrical lead 12 further comprises an electrode array 20 comprising a plurality of electrodes 22. Electrode array 20 is located on distal portion 16 of lead body 14. Although lead 12 is depicted in FIG. 1 as a percutaneous elongated lead with electrodes disposed circumferentially about the distal portion of the lead, the lead could also be a paddle-style lead as depicted in FIG. 6 and as described in co-pending application entitled: “Electrical Lead Placement System” (Ref. No. NAT-021915-US-ORD), filed on Mar. 15, 2013 and incorporated by reference herein. In particular, such a paddle-style lead 60 comprises an electrical elongate lead body 62 having a proximal end and a distal end 64 and a substantially flat paddle 66. Paddle 66 comprises a paddle body 68 at distal end 64 of lead body 62. Paddle body 68 has a front side 70, a back side 72, a first lateral side 74, a second lateral side 76, a proximal end 78, a distal end 80, and a length extending between the proximal and distal ends 78 and 80. Paddle body 68 comprises an electrode array (not shown) comprising an electrode. The electrode array is located on a portion of the length of front side 70 of paddle body 68.

An electrical lead system further comprises an electrical conductor (not shown) extending between the proximal portion and distal portion 16 of lead body 14 or extending from the proximal end and through the distal end 64 in the case of paddle-style lead 60. The electrical conductor is in electrical communication with electrode array 20 of lead 12 or the electrode array of lead 60.

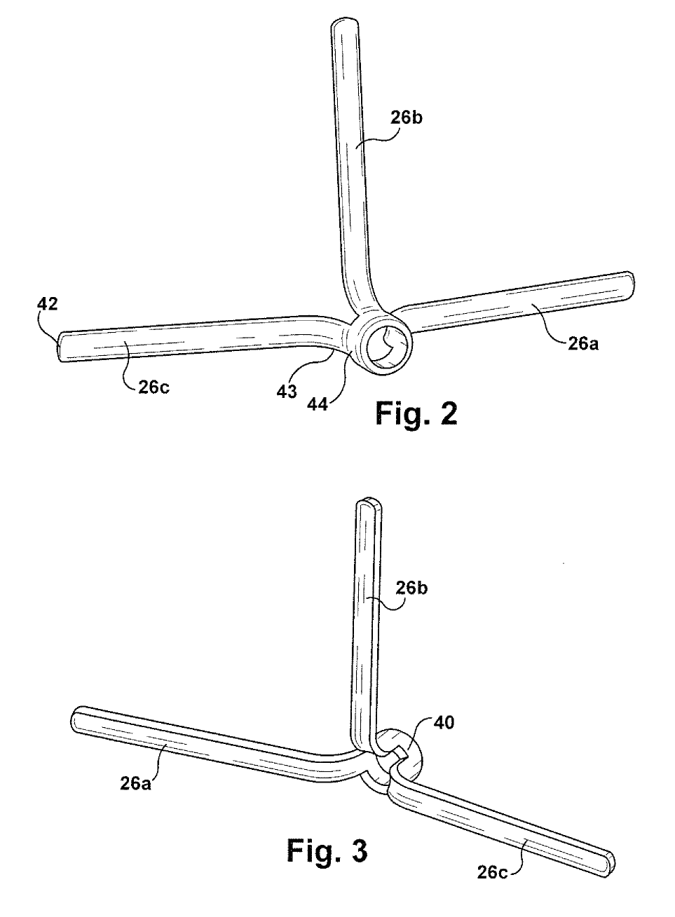

An electrical lead system of the present invention further comprises a positioning device 24 on lead body 14. A positioning device 82 is also on paddle body 68. Although the below disclosure will be described with respect to an exemplary percutaneous lead as depicted in FIG. 1, it is understood that the below disclosure also applies to a paddle-style lead as depicted in FIG. 6, for example, and other leads, catheters and other suitable medical devices. As also shown in FIGS. 2 and 3, the positioning device comprises at least three arms 26 radiating from lead body 14. In this embodiment, the angle between at least two of the arms (such as arm 26a and 26c) is greater than 120 degrees. In certain embodiments, the at least three arms are a first primarily laterally extending arm 26a (also referred to herein as a “laterally extending arm”), a second primarily laterally extending arm 26c (also referred to herein as a “laterally extending arm”), and a primarily posteriorly extending arm 26b (also referred to herein as a “posteriorly extending arm”). Preferably, none of the arms extend in a primarily anterior direction. By “primarily laterally” is meant that the arm is oriented more laterally than any other anatomical direction, such as anteriorly or posteriorly. By “primarily posteriorly” is meant that the arm is oriented more posteriorly than any other anatomical direction such as laterally or anteriorly. By “primarily anterior” is meant that the arm is oriented more anteriorly than any other anatomical direction, such as laterally or posteriorly. In certain embodiments, the first laterally extending arm is separated by about 90 degrees from the posteriorly extending arm, the posteriorly extending arm is separated from the second laterally extending arm by about 90 degrees and the second laterally extending arm is separated from the first laterally extending arm by about 180 degrees. In certain embodiments, the at least three arms is three arms.

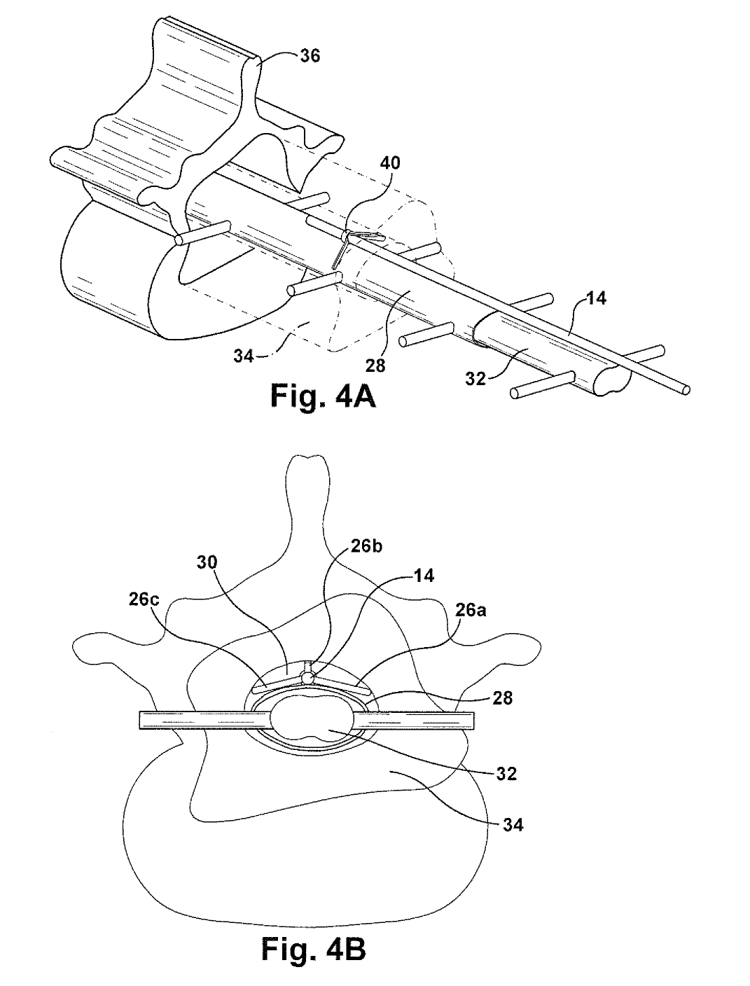

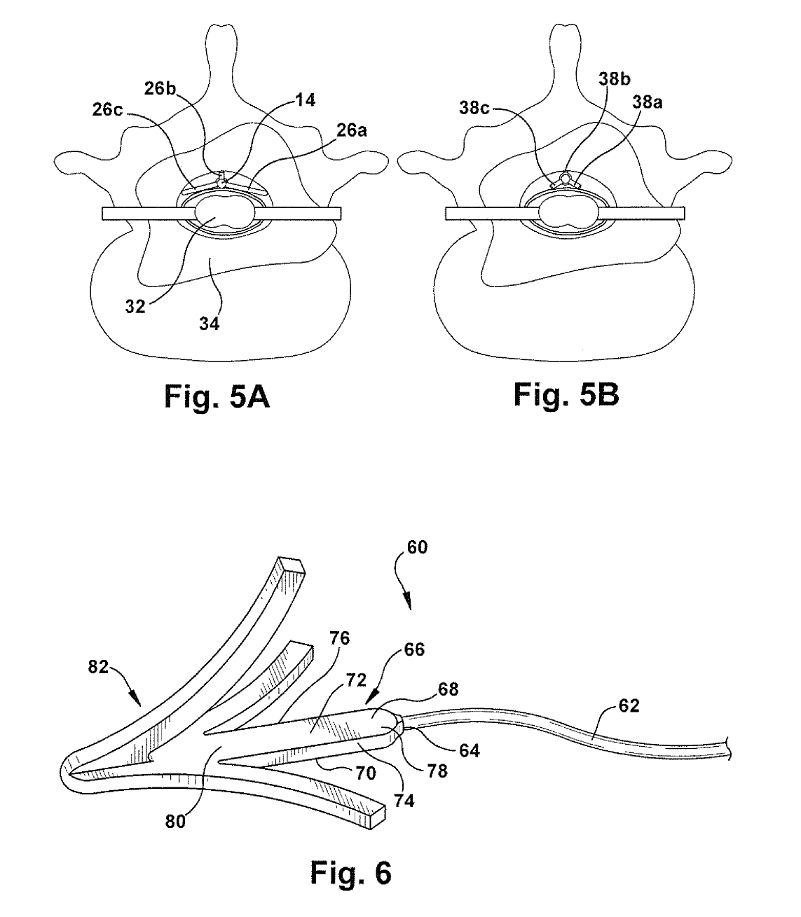

FIGS. 4A and 4B are schematic illustrations of a vertebra 36, a spinal cord 32, a spinal canal 30, and tissue 34 surrounding the spinal canal 30. These figures depict an exemplary lead system deployed in spinal canal 30 where there is greater than a 120 degree spacing between laterally extending arms 26a and 26c. In this embodiment, lead body 14 is inserted into spinal canal 30 and placed on the dura 28 (i.e. epidural positioning). As can be seen from FIG. 5A, the tips of laterally extending arms 26a and 26c and posteriorly extending arm 26b (which are circled in the figure) of the positioning device push off of the respective lateral and posterior tissue interfaces during positioning of the lead to drive laterally extending arms 26a and 26c towards dura 28 and therefore towards spinal cord 32. This results in less energy needed to supply the electrical lead and also provides focal stimulation of the therapy site. In comparison, as illustrated in FIG. 5B, devices that constitute three equally spaced arms radiating from a lead body that are similarly placed on the dura result in a gap between the lead body and dura. The tips of the anteriorly directed arms 38a and 38c may push off the dura instead of pushing off the surrounding tissue, as positioning devices of the present invention do in an operative configuration and as shown in FIG. 5A.

A positioning device of the present invention can be disposed on the lead body in various locations and an electrical lead system of the present invention can include a plurality of positioning devices. Preferably, a positioning device is positioned on the distal portion of the lead body. Further, as shown in FIG. 1, a positioning device can be located distal to the most proximal one of the plurality of electrodes. In other embodiments, a positioning device can be located between adjacent electrodes of the electrode array as shown in FIG. 1. In other embodiments, an electrical lead system includes only one positioning device, which is located proximal to the most proximal one of the plurality of electrodes. In still other embodiments, an electrical lead system includes two positioning devices, the first positioning device located on the lead body distal to the most proximal one of the plurality of electrodes and the second positioning device located on the lead body proximal to the most proximal one of the plurality of electrodes.

The at least three arms of a positioning device of the present invention can be directly/integrally coupled to the lead body as shown in FIG. 6 or can be indirectly coupled to the lead body as shown in FIG. 1. For example, referring to FIGS. 1 and 3, an electrical lead system can further comprise a hub 40 attached to lead body 14 and the plurality of arms 26 can extend from hub 40. In certain embodiments, the hub is operably mountable on the lead body. By “operably mountable” is meant that the positioning device is not pre-assembled on the lead body but is loaded onto the lead body prior to lead insertion and implantation by a user employing a suitable installation tool. The hub can be fabricated from any suitable material such as an elastic material. In such an embodiment, the hub is preferably positioned on the lead body via an elastic interference fit. In certain embodiments, the arms of the positioning device are integrally attached to the hub. In such embodiments, the hub and arms can be fabricated from the same material. In embodiments where the positioning device is directly coupled to the lead body, the positioning device can be integral with, such as integrally molded to, the lead body as depicted in FIG. 6. By “integral” and “integrally molded” is meant that the described components are molded as one piece during manufacturing or the described components are otherwise not separable using a normal amount of force without damaging the integrity (i.e. tearing) either the either component. A normal amount of force is the amount of force a user would use to remove a component, such as a positioning device meant to be separated from the other component, such as a lead, without damaging either structure.

In certain embodiments, each of the arms of a positioning device has a length extending between a free end 42 and an end 43 attached to the lead body or to a hub (as shown in FIG. 2). Preferably, the length is at least one millimeter. Preferably, the laterally extending arms are long enough to contact and push off the lateral tissue interface surrounding the spinal canal in an operative configuration as described above. Similarly, the posteriorly extending arm should preferably be long enough to contact and push off the posterior tissue interface surrounding the spinal canal in an operative configuration as described above.

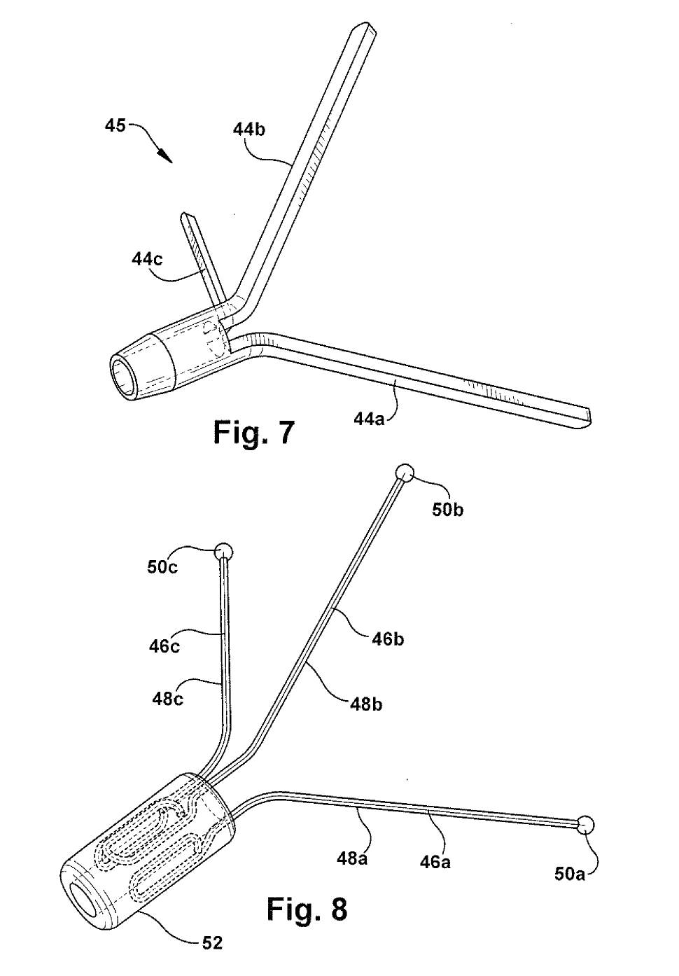

Referring to FIG. 7, in certain embodiments, the arms 44 of a positioning device 45 are fabricated from a material that provides resistance to tissue along the passage of lead implantation but are flexible enough to prevent unintended tissue damage. For example, arms 44 can be fabricated from silicone. Further, the arms can have a blunted shape with no sharp edges as shown in FIG. 7 to also avoid tissue damage. In certain embodiments, arms of a positioning device are fabricated from a material that has greater stiffness than silicone (less that approximately 90 A) to provide the implanted lead with outward force that is resistive to movement or migration. In a preferred embodiment, the arms are made from a material with greater stiffness than silicone, but with sufficient elasticity, such as nitinol. Arms fabricated from nitinol or a material with similar stiffness, may have features to avoid the potential for unintended tissue damage that comes with greater stiffness. Referring to FIG. 8, arms 46 are overmolded with a softer plastic encasement 48 such as urethane or silicone. In addition or alternatively, arms 46 could have substantially spherical atraumatic free ends 50, to avoid having sharp protrusions. As shown in FIG. 8, arms 46 may be formed from nitinol wire having a distal loop-like configuration molded into hub 52. The distal ends of arms 46 could also be secured in hub 52 by other means such as welding to a secondary support band around the hub or by using an adhesive to adhere the distal ends to the hub.

In preferred embodiments, a positioning device is capable of being visualized under fluoroscopy to aid with placement of the positioned device in the spinal canal. For example, radiopaque markings could be incorporated into the positioning device to assist in identification of user-positioned orientation. Such markings include any suitable radiopaque material or impregnation, for example. The markings can be incorporated into the arms and/or hub (if a hub is present) of the positioning device. In other embodiments, markings may be incorporated into an arm that is a different length than the other arms so that a user can determine the direction in which the asymmetrically spaced arm is pointed. In another embodiment, only one arm is impregnated with a radiopaque material, such as barium, so that a user can determine the orientation of the positioning device. Other methods of assisting the user in determining the rotational orientation of the lead and positioning device may be employed.

In certain embodiments, the positioning device is sized to pass through a 14 gauge or greater introducer. Further, in certain embodiments, the system excludes sutures to anchor a positioning device to the lead or to the target site of the patient's body.

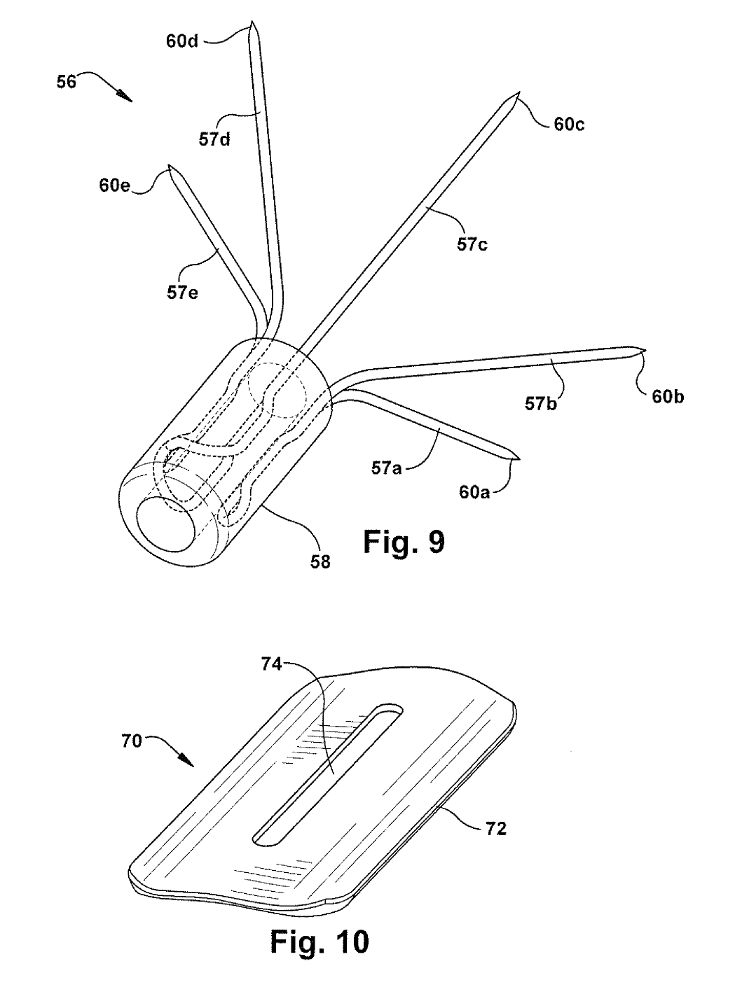

Referring to FIG. 9, the present invention also provides an electrical lead system comprising an anchoring device 56. Such a device comprises a barb 57, and preferably a plurality of barbs 57 that radiate directly from the lead body or radiate indirectly via a hub 58 coupled to the lead body. The anchoring device can be positioned on any suitable location of the lead body but in preferred embodiments, the anchoring device is positioned on the mid-portion of the lead body. The barbs of the anchoring device can be fabricated from any suitable material that allows the barbs to anchor the lead into tissue. For example, the barbs could be fabricated from nitinol. Preferably, as seen in FIG. 9, the free ends 60 of barbs 57 are sharp enough to pierce tissue to hold the lead relative to its implanted position in the body. Preferably, the anchoring device comprises a plurality of barbs spaced circumferentially around the hub or lead body. An anchoring device can be added to a lead after an introducer needle is withdrawn from the lead and the patient. The anchoring device can be added to the lead's outer diameter at the point where the lead enters the tissue incision and can be forced into contact with tissue. Preferably, the barbs bend gently backwards without permanent deformation such that the positioning device may be inserted further into tissue but retraction forces from the proximal end cause the barbs to catch in tissue and effectively protect the lead from proximal pull forces. In certain embodiments, the plurality of barbs comprises at least two barbs extending in opposite directions.

An exemplary method of using a positioning device will now be described. The method comprises providing an electrical lead system. The electrical lead system comprises an electrical lead including an electrical lead body having a proximal portion with a proximal end, a distal portion with a distal end, and a length between the proximal and distal ends. The electrical lead also includes an electrode array comprising a plurality of electrodes located on the distal portion of the lead body. The electrical lead system further comprises an electrical conductor extending between the proximal portion and the distal portion of the lead body and in electrical communication with the electrode array. The electrical lead system also includes a positioning device mountable on the lead body. The positioning device comprises at least three arms radiating from the lead body, wherein the angle between at least two of the arms is greater than 120 degrees. The method further comprises using an installation tool to mount a positioning device on the lead body. The method also includes inserting the electrical lead into the patient's body and placing the electrical lead in a target site of the patient's spinal canal. The at least three arms of the positioning device extends radially outward away from the lead body and offers resistance to tissue in the spinal canal and directs at least one of the electrodes of the electrode array towards the spinal cord. In certain embodiments, the electrical lead in placed in the patient's epidural space and in other embodiments, the lead is placed in the patient's intradural space.

In other embodiments, the present invention provides methods of controlling the delivery of electrical energy to a therapy site in the body. An exemplary method comprises providing an electrical lead system. The electrical lead system comprises an electrical lead including an electrical lead body having a proximal portion with a proximal end, a distal portion with a distal end, and a length between the proximal and distal ends. The electrical lead also includes an electrode array comprising a plurality of electrodes located on the distal portion of the lead body. The electrical lead system further comprises an electrical conductor extending between the proximal portion and the distal portion of the lead body and in electrical communication with the electrode array. The method further comprises providing an insulating device comprising an insulative body defining an aperture therethrough. The method further comprises inserting the electrical lead and insulating device into the patient's body and placing the insulating device between the therapy site and the electrical lead. The method includes aligning the aperture of the insulating device with the therapy site and activating the electrical lead to deliver electrical energy to the therapy site through the aperture of the insulating device. The separate insulating device used in conjunction with an electrical lead essentially controls where electrical current can pass into nerve tissue such as the spinal cord. As shown in FIG. 10, an insulating device 70 can comprise a shield-like insulative body 72 that is substantially flat with an aperture 74 in the form of an elongated slit. Although only one aperture is shown, insulative body 72 can comprise more than one aperture and the shape of the aperture is not limited to an elongated slit. The aperture can have any suitable configuration so long as it allows selective delivery of electrical current. As mentioned above the insulative device is placed between the therapy site, such as the spinal cord, and the electrical lead.

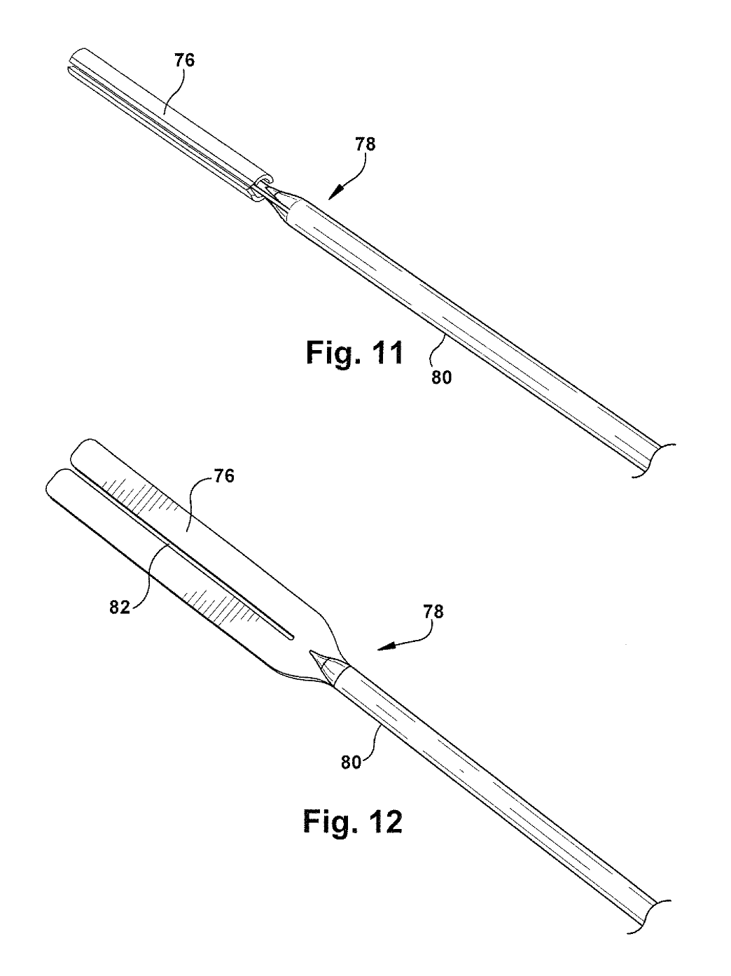

Referring to FIGS. 11 and 12, an insulative body 76 of an insulating device 78 can be expanded and extend distally from a shaft 80. Insulative body 76 defines an aperture 82 that can be an elongated slit. The expandable insulative body can be rollable such that it can be rolled over a lead and positioned between the lead and the therapy site.

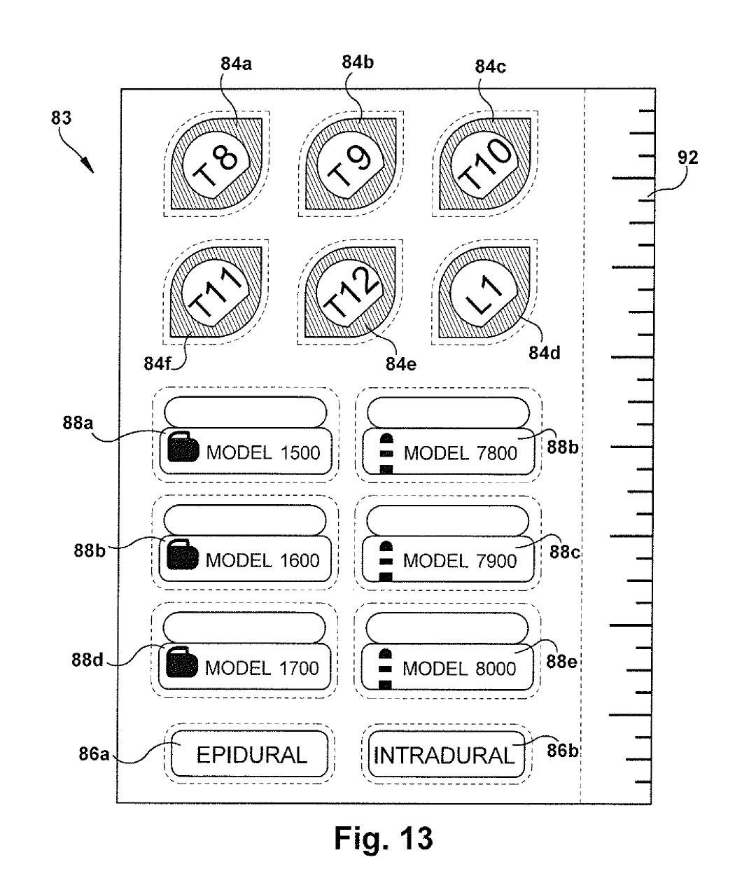

The present invention also provides a fluoroscopic marker system to assist a physician or other health care provider in determining certain features of a implanted medical device, such as where the medical device has been implanted, inserted or advanced; what type of medical device has been implanted, inserted or advanced; the route of insertion; and other information. Referring to FIG. 13, a fluoroscopic marker system 83 according to an embodiment of the present invention comprises a first fluoroscopic label 84 corresponding to a target site of a patient's body. Label 84 comprises a front side and a back side. The front side comprises a radiopaque marking of the name of the target site at which a medical device is implanted in a patient. The back side comprises an adhesive. The first fluoroscopic label 84 is configured for placement on an exterior site of a patient's body. The embodiment shown in FIG. 13 relates to electrical leads implanted for SCS, but it is understood that the marker system could be used for other medical procedures.

In particular with reference to SCS, first fluoroscopic label 84 can having a marking indicating the spinal segment level at which an electrical lead is implanted into a patient. The back side of the label can be configured for placement on a patient's back. Flouroscopic marker system 83 can also include a second fluoroscopic label 86 corresponding to the route of delivery or method of implantation of the medical device. For example, in the case of SCS, second fluoroscopic label 86 can correspond to either the epidural space or the intradural space. As with label 84, second fluoroscopic label 86 comprises a front side and a back side. The front side comprises a radiopaque marking indicating the route of delivery or method of implantation, such as whether an electrical lead has been implanted in the epidural space or the intradural space of a patient in the case of SCS. The back side is similar to the back side of label 84.

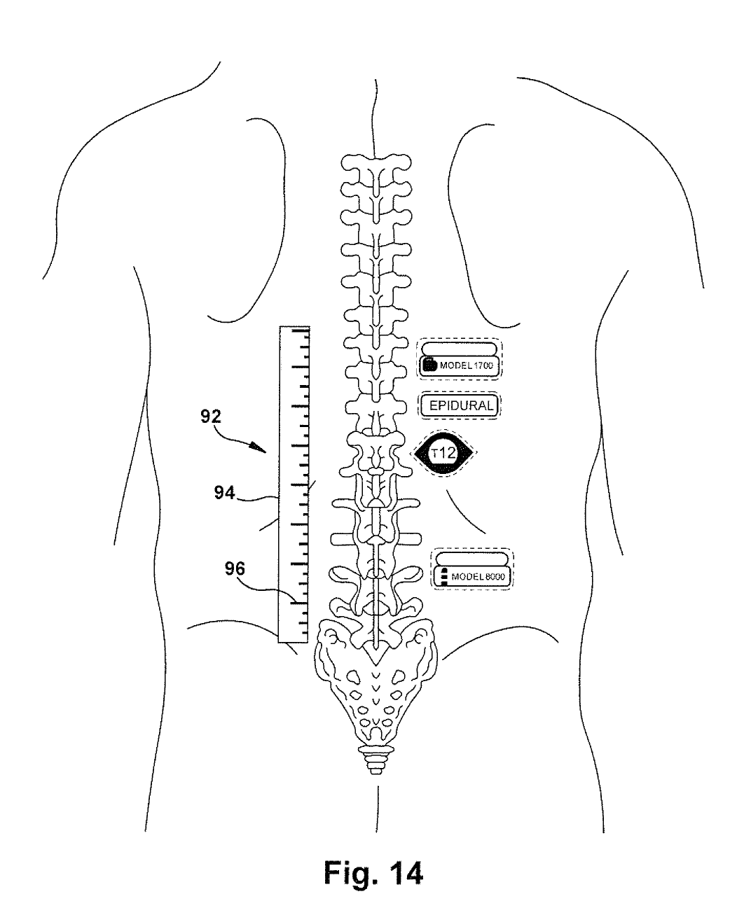

Other fluoroscopic labels can be used as well, such as labels 88 indicating the model number of the medical device, such as the model of the electrical lead, and any accessory devices, such as the model of a generator used to deliver electrical energy to the lead. Referring to FIG. 14, a fluoroscopic label 92 can correspond to a measurement device 94 having radiopaque markings of measurement gradations 96.

The foregoing description and examples have been set forth merely to illustrate the invention and are not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. An electrical lead system for positioning an electrical lead at a target site of a patient's body, the system comprising:
an electrical lead comprising:
an electrical lead body having a proximal portion with a proximal end, a distal portion with a distal end, and a length between the proximal and distal ends, the length having a longitudinal axis;
an electrode array comprising a plurality of electrodes, the electrode array located on the distal portion of the lead body; and
an electrical conductor extending between the proximal portion and the distal portion of the lead body and in electrical communication with the electrode array; and
a positioning device on the lead body, the positioning device comprising at least three arms radiating from the lead body, wherein the angle between at least two of the arms is greater than 120 degrees.

2. The electrical lead system of claim 1, wherein the positioning device is located on the distal portion of the lead body.

3. The electrical lead system of claim 1, wherein the at least three arms comprise a first primarily laterally extending arm, a second primarily laterally extending arm, and no primarily anteriorly extending arm.

4. The electrical lead system of claim 3, wherein one of the at least three arms is a primarily posteriorly extending arm.

5. The electrical lead system of claim 4, wherein the first primarily laterally extending arm is separated by about 90 degrees from the primarily posteriorly extending arm, the primarily posteriorly extending arm is separated from the second primarily laterally extending arm by about 90 degrees, and the second primarily laterally extending arm is separated from the first primarily laterally extending arm by about 180 degrees.

6. The electrical lead system of claim 1, further comprising a hub attached to the lead body, the plurality of arms extending from the hub.

7. The electrical lead system of claim 1, wherein the distal portion of the lead body comprises a paddle-like shape.

8. The electrical lead system of claim 1, wherein the distal portion of the lead body comprises an elongated shape.

9. The electrical lead system of claim 1, wherein the at least three arm is three arms.

10. The electrical lead system of claim 1, wherein the positioning device is distal to the most proximal one of the plurality of electrodes.

11. The electrical lead system of claim 1, wherein the positioning device is only one positioning device proximal to the most proximal one of the plurality of electrodes.

12. The electrical lead system of claim 1, wherein the positioning device is located between adjacent electrodes of the electrode array.

13. The electrical lead system of claim 1, wherein the positioning device is two positioning devices, the first positioning device on the lead body distal to the most proximal one of the plurality of electrodes and the second positioning device on the lead body proximal to the most proximal one of the plurality of electrodes.

14. The electrical lead system of claim 1, further comprising an anchor device on the mid-portion of the lead body, the anchor device comprising a barb radiating from the lead body.

15. The electrical lead system of claim 14, wherein the barb comprises at least two barbs oriented in opposite directions.

16. The electrical lead system of claim 1, wherein the positioning device is a plurality of positioning devices.

17. The electrical lead system of claim 6, wherein the hub of the positioning device is operably mountable on the lead body.

18. The electrical lead system of claim 1, wherein an arm of the at least three arms is radiopaque.

19. The electrical lead system of claim 1, wherein each of the at least three arms has a free end and an end attached to the lead body, an arm of the at least three arms having a rounded atraumatic free end.

20. The electrical lead system of claim 1, wherein an arm of the at least three arms comprises an elastic material.

21. The electrical lead system of claim 1, wherein an arm of the at least three arms is fabricated from nitinol and is coated with a material having a stiffness less than the stiffness of the nitinol.

22. The electrical lead system of claim 1, wherein the at least three arms comprises a distal portion having a loop-like configuration molded into the hub.

23. The electrical lead positioning system of claim 1, wherein the system excludes sutures to anchor the positioning device to the lead, or to anchor the lead to the target site of the patient's body, or both.

24. A method of positioning an electrical lead in a target site of the spinal canal of a patient comprising:
providing an electrical lead system comprising:
an electrical lead comprising:
an electrical lead body having a proximal portion with a proximal end, a distal portion with a distal end, and a length between the proximal and distal ends, the length having a longitudinal axis;
an electrode array comprising a plurality of electrodes, the electrode array located on the distal portion of the lead body; and
an electrical conductor extending between the proximal portion and the distal portion of the lead body and in electrical communication with the electrode array;
using an installation tool to mount a positioning device on the lead body, the positioning device comprising at least three arms radiating from the lead body, wherein the angle between at least two of the arms is greater than 120 degrees;
inserting the electrical lead into the patient's body; and
placing the electrical lead in a target site of the patient's spinal canal, wherein the at least three arms of the positioning device extends radially outward away from the lead body and offers resistance to tissue in the spinal canal and directs at least one of the electrodes of the electrode array towards the spinal cord.

25. The method of claim 24, wherein the target site is the epidural space.

26. The method of claim 24, wherein the target site is the intradural space.

27. A method of controlling the delivery of electrical energy to a therapy site in a patient's body comprising:
providing an electrical lead system comprising:
an electrical lead body having a proximal portion with a proximal end, a distal portion with a distal end, and a length between the proximal and distal ends, the length having a longitudinal axis;
an electrode array comprising a plurality of electrodes, the electrode array located on the distal portion of the lead body; and
an electrical conductor extending between the proximal portion and the distal portion of the lead body and in electrical communication with the electrode array;
providing an insulating device comprising an insulative body defining an aperture therethrough;
inserting the electrical lead and insulating device into the patient's body;
placing the insulating device between the therapy site of the patient's body and the electrical lead;
aligning the aperture of the insulating device with the therapy site; and
activating the electrical lead to deliver electrical energy to the therapy site through the aperture of the insulating device.

28. The method of claim 27, wherein the insulative body has a substantially flat shape and the aperture is an elongated slit.

29. The method of claim 27, wherein the insulative body is an expandable body extending distally from a shaft, the aperture comprising an elongated slit.

30. The method of claim 28, wherein the expandable body is a rollable body extending distally from the shaft.

31. A fluoroscopic marker system comprising:
a first fluoroscopic label corresponding to a target site of a patient's body, the label comprising a front side and a back side, the front side comprising a radiopaque marking indicating the target site at which a medical device is implanted, being inserted into, or being advanced to in a patient, and the back side or the front side comprising an adhesive, the first fluoroscopic label configured for placement on an exterior site of a patient's body.

32. The system of claim 31, wherein the target site is a position in the spinal canal at the level of the desired spinal segment of a patient's body, the medical device is an electrical lead, and the exterior site is the patient's back.

33. The fluoroscopic marker system of claim 32, further comprising a second fluoroscopic label corresponding to either the epidural space or the intradural space, the second fluoroscopic label comprising a front side and a back side, the front side comprising a radiopaque marking indicating whether an electrical lead lead has been implanted in the epidural space or the intradural space of a patient, the back side or the front side comprising an adhesive, the second fluoroscopic label configured for placement on the patient's back.

34. The fluoroscopic marker system of claim 31, further comprising a third fluoroscopic label corresponding to a measurement device, the third fluoroscopic label comprising a front side and a back side, the front side comprising a radiopaque marking of measurement gradations, the back side or the front side comprising an adhesive, the third fluoroscopic label configured for placement on an exterior site of a patient's body.

35. The system of claim 34, wherein the exterior site is the patient's back.

36. The fluoroscopic marker system of claim 31, further comprising a fourth fluoroscopic label corresponding to a model of the medical device implanted, the fourth fluoroscopic label comprising a front side and a back side, the front side comprising a radiopaque marking indicating the medical device model, the back side or the front side comprising an adhesive, the fourth fluoroscopic label configured for placement on an exterior site of a patient's body.

37. The fluoroscopic marker system of claim 34, further comprising a fourth fluoroscopic label corresponding to one or more of a model of an electrical lead implanted in a patient or a model of a generator implanted in a patient, the fourth fluoroscopic label comprising a front side and a back side, the front side comprising a radiopaque marking indicating one or more of the lead model or the generator model, the back side comprising an adhesive, the fourth fluoroscopic label configured for placement on a patient's back.
